(19) United States
(12) Patent Application Publication
(10) Pub. No.: US 2018/0312838 A1
(43) Pub. Date: Nov. 1, 2018

(54) MODULATION OF MICRORNA 184 TO TREAT PATHOLOGICAL LYMPHANGIOGENESIS

(71) Applicant: The Regents of the University of California, Oakland, CA (US)

(72) Inventors: Lu Chen, Oakland, CA (US); Sammy Grimaldo, Oakland, CA (US)

(73) Assignee: The Regents of the University of California, Oakland, CA (US)

(21) Appl. No.: 15/769,645

(22) PCT Filed: Oct. 20, 2016
(86) PCT No.: PCT/US2016/057838

§ 371 Date: Apr. 19, 2018

Related U.S. Application Data
(60) Provisional application No. 62/244,542, filed on Oct. 21, 2015.

(51) Int. Cl. C12N 15/113; A61P 37/06; A61K 9/00; C12N 7/00

(52) U.S. Cl. CPC C12N 15/113; A61P 37/06; A61K 9/0048; A61K 9/0019; A61K 9/0014; C12N 2320/32; C12N 2710/10043; C12N 2750/14143; C12N 2310/141; C12N 2320/35; C12N 7/00

(57) ABSTRACT

The present invention provides in certain embodiments a method of suppressing pathological lymphatic formation in a tissue or organ in a mammal in need thereof comprising administering a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic.

### CROSS-REFERENCE TO RELATED APPLICATION

This application claims priority to U.S. Provisional Application No. 62/244,542 that was filed on Oct. 21, 2015. The entire content of this provisional application referenced above is hereby incorporated by reference herein.

### GOVERNMENT FUNDING

This invention was made with government support under EY017392 awarded by the National Institutes of Health. The government has certain rights in the invention.

### BACKGROUND OF THE INVENTION

The lymphatic network penetrates most tissues and its dysfunction is associated with a broad spectrum of disorders, such as cancer metastasis, inflammation, transplant rejection, hypertension, obesity, and lymphedema (Alitalo K. The lymphatic vasculature in disease. Nature medicine 2011; 17:1371-1380; Chen L. Ocular lymphatics: state-of-the-art review. Lymphology 2009; 42:66-76). After being neglected for centuries due to historical reasons and technical limitations, lymphatic research has gained significant attention and great progress in recent years. However, currently, there is no effective treatment for lymphatic diseases in the eye. It is therefore imperative to identify new regulators of lymphangiogenesis (LG; the formation of lymphatic vessels) in order to develop novel therapeutic strategies.

### SUMMARY

The present invention provides in certain embodiments a method of suppressing pathological lymphatic formation in a tissue or organ in a mammal in need thereof comprising administering a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic.

The present invention provides in certain embodiments a method of inhibiting cancer metastasis in a mammal in need thereof comprising administering a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic.

The present invention provides in certain embodiments a method of inhibiting transplant rejection in a mammal in need thereof comprising administering a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic.

The present invention provides in certain embodiments a method of inhibiting adhesion, migration and/or tube formation of lymphatic endothelial cells (LECs) comprising administering to a mammal in need thereof a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic, wherein the inhibition is by about 10% as compared to non-treated LECs. In certain embodiments, the inhibition is by at least 40%.

The present invention provides in certain embodiments a method of preventing or treating corneal lymphangiogenesis (LG) in a mammal in need thereof comprising administering a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic. In certain embodiments, the corneal lymphangiogenesis is induced by inflammation, infection, dry eye, trauma, or chemical damage.

The present invention provides in certain embodiments a method of modifying a cornea before or after transplantation to improve graft survival comprising administering a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic.

In certain embodiments, the tissue is eye tissue.

In certain embodiments, the eye tissue is corneal tissue.

In certain embodiments, the tissue is endothelial tissue.

In certain embodiments, the endothelial tissue is of lymphatic endothelial tissue.

In certain embodiments, the mammal is a human.

In certain embodiments, the therapeutic agent is present within a pharmaceutical composition.

In certain embodiments, the administration is by local or systemic administration.

In certain embodiments, the administration is by subconjunctival, intraocular, periocular, retrobulbar, intramuscular, topical, intravenous, or subcutaneous administration.

In certain embodiments, the agent is a Mir-184 mimic that is 22 nucleotides in length.

In certain embodiments, the agent is a pri-Mir-184 from 100-200 bp in length.

In certain embodiments, the agent is a Mir-184 mimic that has at least 90% complementarity to SEQ ID NO: 1.

In certain embodiments, the agent is a vector and the promoter is a polII or polIII promoter.

In certain embodiments, the agent is a vector and the promoter is an H1 or U6 promoter.

In certain embodiments, the agent is a vector and the promoter is a tissue-specific promoter.

In certain embodiments, the agent is a vector and the promoter is an inducible promoter.

In certain embodiments, the agent is a vector is an adeno-associated virus (AAV) vector or adenovirus vector.

The present invention provides in certain embodiments a composition comprising a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic for use in the therapeutic treatment of pathological lymphatic formation in a tissue or organ.

The present invention provides in certain embodiments a use of Mir-184, pri-Mir-184, a Mir-184 mimic, or a vector comprising an expression cassette comprising a promoter and a nucleic acid encoding Mir-184, pri-Mir-184, or a Mir-184 mimic to prepare a medicament useful for inhibiting LG in a mammal.

### BRIEF DESCRIPTION OF THE FIGURES

FIGS. 1A-1C. Mir-184 expression is down-regulated in corneal inflammatory lymphangiogenesis. (FIG. 1A) Real-time PCR results showing mir-184 is significantly down-regulated in the inflamed cornea 2 weeks after suture placement. (FIG. 1B) Representative images of immunofluorescent microscopic analysis showing significantly reduced lymphatic vessels in the inflamed cornea after mir-184 mimic treatment. White dashed line: demarcation of the limbus between the cornea and conjunctiva. Scale bar: 200 pin. Summarized data in terms of lymphatic invasion area are presented in (FIG. 1C). Data are expressed as mean±SEM, * P<0.05.

FIGS. 2A-2B. Overexpression of mir-184 in human lymphatic endothelial cells in vitro. (FIG. 2A) Gel images of mir-184 PCR product 48 hours post-transfection with mir-184 synthetic mimic. (FIG. 2B) Quantitative real-time PCR result showing significant increase of mir-184 in the LECs after transfection. Data are expressed as mean±SEM, * P<0.05.

FIGS. 3A-3C. Mir-184 overexpression inhibits lymphatic endothelial cell functions of adhesion and migration. (FIG. 3A) Summarized data showing significant suppression of LEC adhesion to collagen type I after transfection with mir-184 mimic (FIG. 3B) and (FIG. 3C) Summarized data (FIG. 3B) and representative images (FIG. 3C) showing significant inhibition of LEC migration in a wound healing assay after transfection with mir-184 mimic Scale bar: 500 μm. Data are expressed as mean±SD, * P<0.05.

FIGS. 4A-4B. Mir-184 overexpression inhibits lymphatic endothelial cell tube formation.

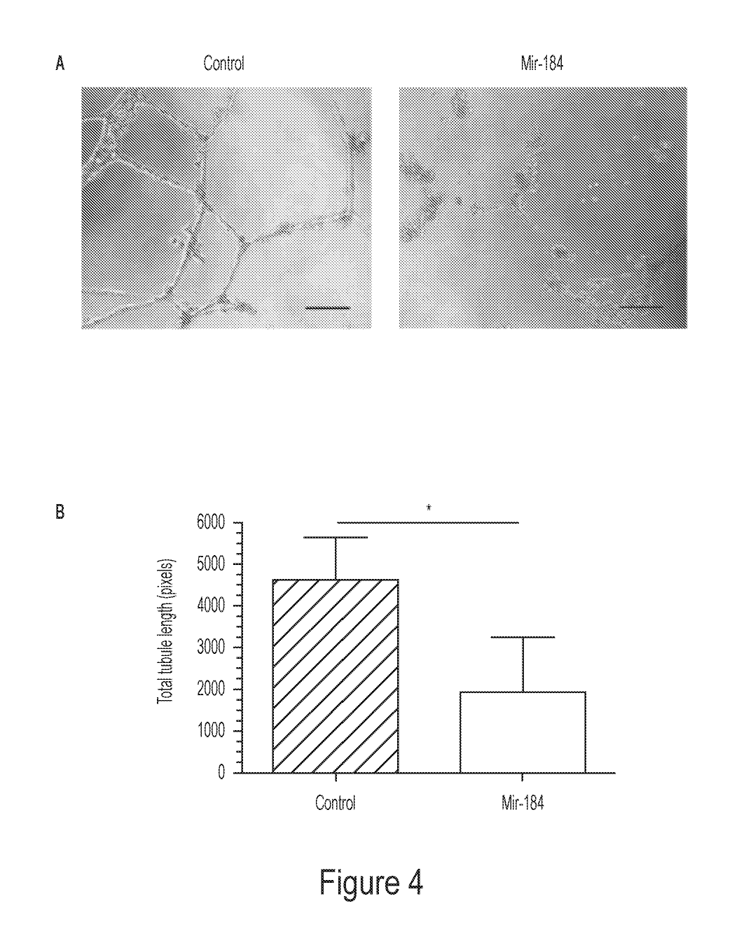

(FIG. 4A) Representative micrographs showing significant inhibition of LEC capillary tube formation on matrigel after transfection with mir-184 mimic Scale bar: 200 μm. (FIG. 4B) Summarized data on total tubule length measurement. Data are expressed as mean±SD, * P<0.05.

### DETAILED DESCRIPTION

Unlike blood vessels which have been studied for centuries in the past, lymphatic research denotes a field of new discovery and has experienced exponential growth in recent years (Alitalo, K. The lymphatic vasculature in disease. Nat. Med. 2011; 17:1371-1380; Tammela T, Alitalo K: Lymphangiogenesis: Molecular mechanisms and future promise. Cell 2010; 140:460-476; Chen L: Ocular Lymphatics: State-of-the-Art Review. Lymphology 2009; 42:66-76; Alitalo K, Tammela T, Petrova T V: Lymphangiogenesis in development and human disease; Nature 2005; 438:946-953; Brown P: Lymphatic system: unlocking the drains. Nature 2005; 436:456-458; Folkman J, Kaipainen A: Genes tell lymphatics to sprout or not. Nat Immunol 2004; 5:11-12; Rockson S G: The broad spectrum of lymphatic health and disease. Lymphat Res Biol; 8:101). The lymphatic network penetrates most tissues in the body and plays critical roles in a broad spectrum of functions, such as immune surveillance, body fluid regulation, and fat and vitamin absorption. Numerous diseases and conditions are therefore associated with lymphatic dysfunction, which include but are not limited to cancer metastasis, tissue and major organ (heart, kidney and lung) transplant rejection, inflammatory and immune diseases, infections, asthma, obesity, diabetes, AIDS, hypertension and lymphedema. These disorders can be disabling, disfiguring, and even life threatening. To date, there is little effective treatment for lymphatic disorders, so it is a field with an urgent demand for new therapeutic protocols. The burdens associated with lymphatic diseases are immense. For example, lymphedema (primary or secondary to cancer therapy) alone affect at least over 6 million individuals in the United States and more than 170 million people worldwide.

The cornea provides an ideal site for lymphatic research due to its accessible location, transparent nature, and lymphatic-free but—inducible features (Chen L: Ocular Lymphatics: State-of-the-Art Review. Lymphology 2009; 42:66-76; Rogers M S, Birsner A E, D'Amato R J: The mouse cornea micropocket angiogenesis assay. Nat Protoc 2007; 2:2545-2550; Cursiefen C, Chen L, Dana M R, Streilein J W: Corneal lymphangiogenesis: evidence, mechanisms, and implications for corneal transplant immunology. Cornea 2003; 22:273-281; Chen L, Hann B, Wu L: Experimental models to study lymphatic and blood vascular metastasis, in “From local invasion to metastatic cancer”. Stanley P. L. Leong (Editor), Humana Press. 2011). The success of using the cornea for lymphatic research can be predicted from the fact that during past centuries, more than one third of basic knowledge on blood vessels was obtained from studies with the cornea, as estimated by Judah Folkman, the grandfather of tumor angiogenesis research.

Though not supplied by lymphatic vessels under normal condition, lymphatic formation (lymphangiogenesis, LG) accompanies many corneal diseases after an inflammatory, infectious, traumatic, immunogenic, or chemical damage. Once induced, they enhance high volume delivery of immune cells and mediate transplant rejection as well. Collective data from us and other researchers have shown that the lymphatic pathway is a primary mediator of corneal transplant rejection (Dietrich T, Bock F, Yuen D, et al.: Cutting edge: lymphatic vessels, not blood vessels, primarily mediate immune rejections after transplantation. J Immunol 2010; 184:535-539; Zhang H, Grimaldo S, Yuen D, Chen L: Combined blockade of VEGFR-3 and VLA-1 markedly promotes high-risk corneal transplant survival. Invest Ophthalmol Vis Sci 2011). Moreover, LG has been associated with transplant rejection in other parts of the body (such as kidney, heart, lung, bone implant) (Kerjaschki D: Lymphatic neoangiogenesis in renal transplants: a driving force of chronic rejection? J Nephrol 2006; 19:403-406; Kerjaschki D, Huttary N, Raab I, et al.: Lymphatic endothelial progenitor cells contribute to de novo lymphangiogenesis in human renal transplants. Nat Med 2006; 12:230-234; Jell G, Kerjaschki D, Revell P, Al-Saffar N: Lymphangiogenesis in the bone-implant interface of orthopedic implants: importance and consequence. J Biomed Mater Res A 2006; 77:119-127; Geissler H J, Dashkevich A, Fischer U M, et al.: First year changes of myocardial lymphatic endothelial markers in heart transplant recipients. Eur J Cardiothorac Surg 2006; 29:767-771).

Corneal transplantation is the most common form among all solid organ and tissue transplantation. Although it enjoys a high survival rate in uninflamed and alymphatic host reds, the rejection rate can be as high as 50-90% when the grafting is performed on inflamed and lymphatic-rich corneas. To date, there is still few effective management of this high rejection situation. Unfortunately, many patients who are blind as a result of corneal diseases fall in this high-rejection category (after a traumatic, inflammatory, infectious, or chemical damage). Due to the poor prognosis, these patients are not even considered as good candidates for the transplantation surgery and have to give up their hope for vision restoration. We are therefore interested in identifying new therapeutic targets to treat LG and its related disorders including graft rejection. The cornea offers an ideal site for LG research. Due to its accessible location, transparent nature, and alymphatic feature under normal condition, this tissue provides a favorable model to study inducible lymphatic growth without having to distinguish from pre-existing or background vessels (Chen L. Ocular lymphatics: state-of-the-art review. Lymphology 2009; 42:66-76). Corneal L G can be induced by a number of pathological insults, such as inflammation, infection, trauma, and chemical burns, and it is a primary mediator of transplant rejection (Chen L. Ocular lymphatics: state-of-the-art review. Lymphology 2009; 42:66-76; Dietrich T, Bock F, Yuen D, et al. Cutting edge: lymphatic vessels, not blood vessels, primarily mediate immune rejections after transplantation. J Immunol 2010; 184:535-539; Yuen D, Pytowski B, Chen L. Combined blockade of VEGFR-2 and VEGFR-3 inhibits inflammatory lymphangiogenesis in early and middle stages. Invest Ophthalmol Vis Sci 2011; 52:2593-2597).

This invention has a number of potential applications, such as preventing or treating corneal lymphangiogenesis, which is induced by inflammation, infection, dry eye, trauma, or chemical damage; modifying the cornea before and after transplantation to improve graft survival; suppressing lymphangiogenesis and promote transplant survival in other tissues or organs of the body; or managing lymphatic disorders occurring inside and outside the eye (such as cancer metastasis, inflammatory and immune diseases, and lymphedema).

Currently, there is no effective treatment for lymphatic diseases in the eye. Mir-184 is normally present in the alymphatic cornea and thus provides a novel and natural inhibitor of lymphangiogenesis. The pharmacotherapy of corneal graft rejection has changed little over the past decades despite the fact that corticosteroids is of limited efficacy and is fraught with serious side effects, such as glaucoma, cataracts, and opportunistic infections. The clinical burden of graft rejection in inflamed and lymphatic-rich grafting bed is tremendous, since as high as 50-90% of the grafts are rejected, irrespective of current treatment modalities.

MicroRNAs are a class of small non-coding RNAs that regulate gene expression by RNA silencing and post-transcriptional regulation (Hammond S M. An overview of microRNAs. Advanced drug delivery reviews 2015; Ambros V. The functions of animal microRNAs. Nature 2004; 431:350-355). Their specific roles in the eye and eye-related diseases still remain largely unknown. A recent study using miRNA arrays to compare adult mouse cornea to epithelial-rich footpads has identified microRNA-184 (mir-184) as the most abundantly expressed microRNA in the mouse cornea (Ryan D G, Oliveira-Fernandes M, Lavker R M. MicroRNAs of the mammalian eye display distinct and overlapping tissue specificity. Mol Vis 2006; 12:1175-1184). The restrictive expression profile of mir-184 in the normal and alymphatic cornea has prompted us to evaluate its potential role in corneal LG and whether it can be used as an anti-lymphangiogenic factor (Ryan D G, Oliveira-Fernandes M, Lavker R M. MicroRNAs of the mammalian eye display distinct and overlapping tissue specificity. Mol Vis 2006; 12:1175-1184; Karali M, Peluso I, Marigo V, Banfi S. Identification and characterization of microRNAs expressed in the mouse eye. Invest Ophthalmol Vis Sci 2007; 48:509-515).

The present inventors have discovered the novel finding that microRNA-184 (Mir-184) is critically involved in pathological lymphatic vessel formation. Mir-184 expression is significantly down-regulated in corneal inflammatory lymphangiogenesis, and its synthetic mimics suppress corneal lymphatic growth in vivo. Moreover, Mir-184 ectopic expression in human microdermal lymphatic endothelial cells inhibits critical cell functions of adhesion, migration, and tube formation in vitro. These data indicate Mir-184 as an inhibitor of lymphangiogenesis and its modulation is used as a new strategy to regulate lymphatic vessel growth and to treat lymphatic-related diseases of the eye (such as inflammation, infection, dry eye, and corneal transplant rejection). Alternatively, it is also indicated that anti-Mir-184 strategy could be used to promote lymphatic formation and to treat lymphatic deficiency disorders, such as lymphedema.

This invention is important because: 1) pathological lymphangiogenesis is induced by many diseases after an inflammatory, infectious, immunogenic, toxic, traumatic, or chemical insults; 2) lymphangiogenesis, once induced, is a primary mediator of transplant rejection. The clinical burden of graft rejection in the high-risk transplantation is tremendous, since as high as 50-90% of the grafts are rejected irrespective of current treatment modalities. The pharmacotherapy of transplant rejection has changed little over the past decades despite the fact that corticosteroids is of limited efficacy and is fraught with serious side effects, such as glaucoma, cataracts, and opportunistic infections; 3) Mir-184 is normally expressed in lymphatic-free cornea and functions as a natural inhibitor of lymphangiogenesis; and 4) the combination of both animal and human cell work provide translatable information to patient condition. This strategy provides new therapeutic strategies to treat a number of lymphatic diseases that occur both inside and outside the eye.

The present data indicate that Mir-184 mimics can be used to suppress pathological lymphatic formation in the cornea. Mir-184 mimics, or their derivatives, can be administrated locally or systemically to treat eye diseases (subconjunctival, intraocular, periocular, retrobulbar, intramuscular, topical, intravenous, subcutaneous, etc.). The invention can also be used to treat other lymphatic disorders in the body, such as cancer metastasis, and inflammatory and immune diseases. Alternatively, anti-Mir-184 strategy may be potentially used to promote lymphatic formation and treat lymphatic deficiency disorders, such as lymphedema. In certain embodiments, lymphangiogenesis is suppressed by at least 10%. In certain embodiments, lymphangiogenesis is suppressed by at least 10%, 15%, 20%, 25%, 30%, 35%, 40%, 45%, 50%, 55%, 60%, 65%, 70%, 75%, 80%, 85%, 90% 95%, 99% or 100% In certain embodiments, lymphangiogenesis is suppressed at a level sufficient to cause a therapeutic effect. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the condition. Therapeutic effects can be measured quantitatively by a physician or qualitatively by a patient afflicted with LG targeted by the miRNA.

In one embodiment, the invention features a method for treating or preventing LG in a subject or organism comprising, contacting the subject or organism with an miRNA molecule of the invention via local administration to relevant tissues or cells, for example, by administration of vectors or expression cassettes of the invention that provide miRNA molecules of the invention to relevant cells.

Methods of delivery of viral vectors include, but are not limited to, intravenous administration and administration directly into a patient's eye. Generally, AAV virions may be introduced into cells using either in vivo or in vitro transduction techniques. If transduced in vitro, the desired recipient cell will be removed from the subject, transduced with AAV virions and reintroduced into the subject. Alternatively, syngeneic or xenogeneic cells can be used where those cells will not generate an inappropriate immune response in the subject.

In one embodiment, pharmaceutical compositions will comprise sufficient genetic material to produce a therapeutically effective amount of the miRNA of interest, i.e., an amount sufficient to reduce or ameliorate symptoms of the disease state in question or an amount sufficient to confer the desired benefit. The pharmaceutical compositions may also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

As is apparent to those skilled in the art in view of the teachings of this specification, an effective amount of viral vector which must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

It should be understood that more than one transgene could be expressed by the delivered viral vector. Alternatively, separate vectors, each expressing one or more different transgenes, can also be delivered as described herein. Furthermore, it is also intended that the viral vectors delivered by the methods of the present invention be combined with other suitable compositions and therapies.

The present invention further provides miRNA, an expression cassette and/or a vector as described herein for use in medical treatment or diagnosis.

The present invention provides the use of an miRNA, an expression cassette and/or a vector as described herein to prepare a medicament useful for treating LG.

The present invention also provides a nucleic acid, expression cassette, vector, or composition of the invention for use in therapy.

The present invention also provides a nucleic acid, expression cassette, vector, or composition of the invention for treating LG.

“Treating” as used herein refers to ameliorating at least one symptom of, curing and/or preventing the development of LG.

The term “nucleic acid” refers to deoxyribonucleic acid (DNA) or ribonucleic acid (RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

A “nucleotide sequence” is a polymer of DNA or RNA that can be single-stranded or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The invention encompasses isolated or substantially purified nucleic acid nucleic acid molecules and compositions containing those molecules. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences are also encompassed by the present invention. By “fragment” or “portion” is meant a full length or less than full length of the nucleotide sequence.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

A “transgene” refers to a gene that has been introduced into the genome by transformation. Transgenes include, for example, DNA that is either heterologous or homologous to the DNA of a particular cell to be transformed. Additionally, transgenes may include native genes inserted into a non-native organism, or chimeric genes. The term “endogenous gene” refers to a native gene in its natural location in the genome of an organism.

“Wild-type” refers to the normal gene or organism found in nature.

A “vector” is defined to include, inter alia, any viral vector, as well as any plasmid, cosmid, phage or binary vector in double or single stranded linear or circular form that may or may not be self-transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. The coding region usually codes for a functional RNA of interest, for example an miRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Regulatory sequences” are nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted herein, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. Examples of promoters that may be used in the present invention include the mouse U6 RNA promoters, synthetic human H1RNA promoters, SV40, CMV, RSV, RNA polymerase II and RNA polymerase III promoters.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of miRNA constructs, expression may refer to the transcription of the miRNA only.

“Altered levels” refers to the level of expression in transgenic cells or organisms that differs from that of normal or untransformed cells or organisms.

“Overexpression” refers to the level of expression in transgenic cells or organisms that exceeds levels of expression in normal or untransformed cells or organisms.

The term “substantial identity” of polynucleotide sequences means that a polynucleotide comprises a sequence that has at least 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells.

“Transformed,” “transduced,” “transgenic” and “recombinant” refer to a host cell into which a heterologous nucleic acid molecule has been introduced. As used herein the term “transfection” refers to the delivery of DNA into eukaryotic (e.g., mammalian) cells. The term “transformation” is used herein to refer to delivery of DNA into prokaryotic (e.g., E. coli) cells. The term “transduction” is used herein to refer to infecting cells with viral particles. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

As used herein, the term “derived” or “directed to” with respect to a nucleotide molecule means that the molecule has complementary sequence identity to a particular molecule of interest.

The miRNAs of the present invention can be generated by any method known to the art, for example, by in vitro transcription, recombinantly, or by synthetic means. In one example, the miRNAs can be generated in vitro by using a recombinant enzyme, such as T7 RNA polymerase, and DNA oligonucleotide templates.

MicroRNAs

MicroRNAs (miRNAs or mirs) are a highly conserved class of small RNA molecules that are transcribed from DNA in the genomes of plants and animals, but are not translated into protein. Pre-microRNAs are processed into miRNAs (Zhang et al., J. Controlled Release 172(2013) 962-974). Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

Single-stranded oligonucleotides, including those described and/or identified as microRNAs or mirs which may be used as targets or may serve as a template for the design of oligonucleotides of the invention. It will be appreciated by one of skill in the art that any oligonucleotide chemical modifications or variations describe herein also apply to single stranded oligonucleotides.

Mature human Mir-184 (hsa-miR-184) has the following sequence:

(SEQ ID NO: 1)
5′-UGGACGGAGAACUGAUAAGGGU-3′

Human Pri-Mir-184 has the following sequence:

(SEQ ID NO: 2)
5′-CCAGUCACGUCCCCUUAUCACUUUUCCAGCCCAGCUUUGUGACUGUA
AGUGUUGGACGGAGAACUGAUAAGGGUAGGUGAUUGA-3′

miRNA Mimics

miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g., single stranded) or mimic precursors (e.g., pri- or pre-miRNAs). miRNA mimics can be comprised of nucleic acid (modified or modified nucleic acids) including oligonucleotides comprising, without limitation, RNA, modified RNA, DNA, modified DNA, locked nucleic acids, or 2′-O,4′-C-ethylene-bridged nucleic acids (ENA), or any combination of the above (including DNA-RNA hybrids). In addition, miRNA mimics can comprise conjugates that can affect delivery, intracellular compartmentalization, stability, specificity, functionality, strand usage, and/or potency. In one design, miRNA mimics are double stranded molecules (e.g., with a duplex region of between about 16 and about 31 nucleotides in length) and contain one or more sequences that have identity with the mature strand of a given miRNA. Modifications can comprise 2′ modifications (including 2′-O methyl modifications and 2′ F modifications) on one or both strands of the molecule and internucleotide modifications (e.g. phorphorthioate modifications) that enhance nucleic acid stability and/or specificity. In addition, miRNA mimics can include overhangs. The overhangs can consist of 1-6 nucleotides on either the 3′ or 5′ end of either strand and can be modified to enhance stability or functionality. In one embodiment, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

MicroRNA Replacement Therapy

miRNA replacement therapy supplements a lowered level of miRNA with oligonucleotide mimics containing the same sequence as the mature endogenous miRNA. Double stranded miRNA mimics compose of a guide strand a passenger strand have 100 to 1000 fold higher potency compared with single stranded miRNA mimics Zhang et al., J. Controlled Release 172(2013) 962-974). The guide strand contains a sequence identical to the mature miRNA and the passenger strand sequence is complementary to the mature miRNA. In addition to the miRNA mimics having identical sequence as the endogenous mature miRNA, synthetic miRNA precursor mimics with longer sequence ranging from just a few additional nucleotides to full length pri-miRNA may be used.

Nucleic Acid Molecules of the Invention

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. The RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

Expression Cassettes of the Invention

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

A “chimeric” vector or expression cassette, as used herein, means a vector or cassette including nucleic acid sequences from at least two different species, or has a nucleic acid sequence from the same species that is linked or associated in a manner that does not occur in the “native” or wild-type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the miRNA, or the like. Such elements may be included in the DNA as desired to obtain the optimal performance of the miRNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein.

The recombinant DNA can be readily introduced into the host cells, e.g., mammalian, bacterial, yeast or insect cells by transfection with an expression vector composed of DNA encoding the miRNA by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described herein below, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As discussed herein, a “transfected” “or “transduced” host cell or cell line is one in which the genome has been altered or augmented by the presence of at least one heterologous or recombinant nucleic acid sequence. The host cells of the present invention are typically produced by transfection with a DNA sequence in a plasmid expression vector, a viral expression vector, or as an isolated linear DNA sequence. The transfected DNA can become a chromosomally integrated recombinant DNA sequence, which is composed of sequence encoding the miRNA.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

The instant invention provides a cell expression system for expressing exogenous nucleic acid material in a mammalian recipient. The expression system, also referred to as a “genetically modified cell,” comprises a cell and an expression vector for expressing the exogenous nucleic acid material. The genetically modified cells are suitable for administration to a mammalian recipient, where they replace the endogenous cells of the recipient. Thus, the preferred genetically modified cells are non-immortalized and are non-tumorigenic.

According to one embodiment, the cells are transfected or otherwise genetically modified ex vivo. The cells are isolated from a mammal (preferably a human), nucleic acid introduced (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

As used herein, “exogenous nucleic acid material” refers to a nucleic acid or an oligonucleotide, either natural or synthetic, which is not naturally found in the cells; or if it is naturally found in the cells, is modified from its original or native form. Thus, “exogenous nucleic acid material” includes, for example, a non-naturally occurring nucleic acid that can be transcribed into an anti-sense RNA, a miRNA, as well as a “heterologous gene” (i.e., a gene encoding a protein that is not expressed or is expressed at biologically insignificant levels in a naturally-occurring cell of the same type). To illustrate, a synthetic or natural gene encoding human erythropoietin (EPO) would be considered “exogenous nucleic acid material” with respect to human peritoneal mesothelial cells since the latter cells do not naturally express EPO. Still another example of “exogenous nucleic acid material” is the introduction of only part of a gene to create a recombinant gene, such as combining an regulatable promoter with an endogenous coding sequence via homologous recombination.

The condition amenable to gene inhibition therapy may be a prophylactic process, i.e., a process for preventing disease or an undesired medical condition. Thus, the instant invention embraces a system for delivering miRNA that has a prophylactic function (i.e., a prophylactic agent) to the mammalian recipient.

Methods for Introducing the Expression Cassettes of the Invention into Cells

The inhibitory nucleic acid material (e.g., an expression cassette encoding miRNA directed to a gene of interest) can be introduced into the cell ex vivo or in vivo by genetic transfer methods, such as transfection or transduction, to provide a genetically modified cell. Various expression vectors (i.e., vehicles for facilitating delivery of exogenous nucleic acid into a target cell) are known to one of ordinary skill in the art.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including calcium phosphate DNA co-precipitation, DEAE-dextran, electroporation, cationic liposome-mediated transfection, tungsten particle-facilitated microparticle bombardment, and strontium phosphate DNA co-precipitation.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

The exogenous nucleic acid material can include the nucleic acid encoding the miRNA together with a promoter to control transcription. The promoter characteristically has a specific nucleotide sequence necessary to initiate transcription. The exogenous nucleic acid material may further include additional sequences (i.e., enhancers) required to obtain the desired gene transcription activity. For the purpose of this discussion an “enhancer” is simply any non-translated DNA sequence that works with the coding sequence (in cis) to change the basal transcription level dictated by the promoter. The exogenous nucleic acid material may be introduced into the cell genome immediately downstream from the promoter so that the promoter and coding sequence are operatively linked so as to permit transcription of the coding sequence. An expression vector can include an exogenous promoter element to control transcription of the inserted exogenous gene. Such exogenous promoters include both constitutive and regulatable promoters.

Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the beta-actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain an regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

Accordingly, the amount of miRNA generated in situ is regulated by controlling such factors as the nature of the promoter used to direct transcription of the nucleic acid sequence, (i.e., whether the promoter is constitutive or regulatable, strong or weak) and the number of copies of the exogenous nucleic acid sequence encoding a miRNA sequence that are in the cell.

In addition to at least one promoter and at least one heterologous nucleic acid sequence encoding the miRNA, the expression vector may include a selection gene, for example, a neomycin resistance gene, for facilitating selection of cells that have been transfected or transduced with the expression vector.

Cells can also be transfected with two or more expression vectors, at least one vector containing the nucleic acid sequence(s) encoding the miRNA(s), the other vector containing a selection gene. The selection of a suitable promoter, enhancer, selection gene, and/or signal sequence is deemed to be within the scope of one of ordinary skill in the art without undue experimentation.

The instant invention provides methods for genetically modifying cells of a mammalian recipient in vivo. According to one embodiment, the method comprises introducing an expression vector for expressing an miRNA sequence in cells of the mammalian recipient in situ by, for example, injecting the vector into the recipient.

Delivery Vehicles for the Expression Cassettes of the Invention

Delivery of compounds into tissues can be limited by the size and biochemical properties of the compounds. Currently, efficient delivery of compounds into cells in vivo can be achieved only when the molecules are small (usually less than 600 Daltons).

The selection and optimization of a particular expression vector for expressing a specific miRNA in a cell can be accomplished by obtaining the nucleic acid sequence of the miRNA, possibly with one or more appropriate control regions (e.g., promoter, insertion sequence); preparing a vector construct comprising the vector into which is inserted the nucleic acid sequence encoding the miRNA; transfecting or transducing cultured cells in vitro with the vector construct; and determining whether the miRNA is present in the cultured cells.

Vectors for cell gene therapy include viruses, such as replication-deficient viruses. Exemplary viral vectors are derived from Harvey Sarcoma virus, ROUS Sarcoma virus, (MPSV), Moloney murine leukemia virus and DNA viruses (e.g., adenovirus).

Replication-deficient retroviruses are capable of directing synthesis of all virion proteins, but are incapable of making infectious particles. Accordingly, these genetically altered retroviral expression vectors have general utility for high-efficiency transduction of nucleic acid sequences in cultured cells, and specific utility for use in the method of the present invention. Such retroviruses further have utility for the efficient transduction of nucleic acid sequences into cells in vivo. Retroviruses have been used extensively for transferring nucleic acid material into cells. Protocols for producing replication-deficient retroviruses (including the steps of incorporation of exogenous nucleic acid material into a plasmid, transfection of a packaging cell line with plasmid, production of recombinant retroviruses by the packaging cell line, collection of viral particles from tissue culture media, and infection of the target cells with the viral particles) are well known in the art.

An advantage of using retroviruses for gene therapy is that the viruses insert the nucleic acid sequence encoding the miRNA into the host cell genome, thereby permitting the nucleic acid sequence encoding the miRNA to be passed on to the progeny of the cell when it divides. Promoter sequences in the LTR region have can enhance expression of an inserted coding sequence in a variety of cell types. Some disadvantages of using a retrovirus expression vector are (1) insertional mutagenesis, i.e., the insertion of the nucleic acid sequence encoding the miRNA into an undesirable position in the target cell genome which, for example, leads to unregulated cell growth and (2) the need for target cell proliferation in order for the nucleic acid sequence encoding the miRNA carried by the vector to be integrated into the target genome.

Another viral candidate useful as an expression vector for transformation of cells is the adenovirus, a double-stranded DNA virus. The adenovirus is infective in a wide range of cell types, including, for example, muscle and endothelial cells.

Adenoviruses (Ad) are double-stranded linear DNA viruses with a 36 kb genome. Several features of adenovirus have made them useful as transgene delivery vehicles for therapeutic applications, such as facilitating in vivo gene delivery. Recombinant adenovirus vectors have been shown to be capable of efficient in situ gene transfer to parenchymal cells of various organs, including the lung, brain, pancreas, gallbladder, and liver. This has allowed the use of these vectors in methods for treating inherited genetic diseases, such as cystic fibrosis, where vectors may be delivered to a target organ.

Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Several approaches traditionally have been used to generate the recombinant adenoviruses. One approach involves direct ligation of restriction endonuclease fragments containing a nucleic acid sequence of interest to portions of the adenoviral genome. Alternatively, the nucleic acid sequence of interest may be inserted into a defective adenovirus by homologous recombination results. The desired recombinants are identified by screening individual plaques generated in a lawn of complementation cells.

Application of miRNA is accomplished by transfection of synthetic miRNAs, in vitro synthesized RNAs, or plasmids expressing miRNAs. More recently, viruses have been employed for in vitro studies and to generate transgenic mouse knock-downs of targeted genes. Recombinant adenovirus, adeno-associated virus (AAV) and feline immunodeficiency virus (FIV) can be used to deliver genes in vitro and in vivo. Each has its own advantages and disadvantages. Adenoviruses are double stranded DNA viruses with large genomes (36 kb) and have been engineered to accommodate expression cassettes in distinct regions.

Adeno-associated viruses have encapsidated genomes, similar to Ad, but are smaller in size and packaging capacity (˜30 nm vs. ˜100 nm; packaging limit of ˜4.5 kb). AAV contain single stranded DNA genomes of the + or the − strand. Eight serotypes of AAV (1-8) have been studied extensively. An important consideration for the present application is that AAV5 transduces striatal and cortical neurons, and is not associated with any known pathologies.

Adeno associated virus (AAV) is a small nonpathogenic virus of the parvoviridae family. AAV is distinct from the other members of this family by its dependence upon a helper virus for replication. In the absence of a helper virus, AAV may integrate in a locus specific manner into the q arm of chromosome 19. The approximately 5 kb genome of AAV consists of one segment of single stranded DNA of either plus or minus polarity. The ends of the genome are short inverted terminal repeats which can fold into hairpin structures and serve as the origin of viral DNA replication. Physically, the parvovirus virion is non-enveloped and its icosohedral capsid is approximately 20 nm in diameter.

Further provided by this invention are chimeric viruses where AAV can be combined with herpes virus, herpes virus amplicons, baculovirus or other viruses to achieve a desired tropism associated with another virus. For example, the AAV4 ITRs could be inserted in the herpes virus and cells could be infected. Post-infection, the ITRs of AAV4 could be acted on by AAV4 rep provided in the system or in a separate vehicle to rescue AAV4 from the genome. Therefore, the cellular tropism of the herpes simplex virus can be combined with AAV4 rep mediated targeted integration. Other viruses that could be utilized to construct chimeric viruses include lentivirus, retrovirus, pseudotyped retroviral vectors, and adenoviral vectors.

Also provided by this invention are variant AAV vectors. For example, the sequence of a native AAV, can be modified at individual nucleotides. The present invention includes native and mutant AAV vectors. The present invention further includes all AAV serotypes.

FIV is an enveloped virus with a strong safety profile in humans; individuals bitten or scratched by FIV-infected cats do not seroconvert and have not been reported to show any signs of disease. Like AAV, FIV provides lasting transgene expression in mouse and nonhuman primate neurons, and transduction can be directed to different cell types by pseudotyping, the process of exchanging the virus's native envelope for an envelope from another virus.

Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene silencing therapy and the optimization of the conditions for insertion of the selected expression vector into the cell, are within the scope of one of ordinary skill in the art without the need for undue experimentation.

In another embodiment, the expression vector is in the form of a plasmid, which is transferred into the target cells by one of a variety of methods: physical (e.g., microinjection, electroporation, scrape loading, microparticle bombardment) or by cellular uptake as a chemical complex (e.g., calcium or strontium co-precipitation, complexation with lipid, complexation with ligand). Several commercial products are available for cationic liposome complexation including Lipofectin™ (Gibco-BRL, Gaithersburg, Md.) and Transfectam™ (Promega®, Madison, Wis.). However, the efficiency of transfection by these methods is highly dependent on the nature of the target cell and accordingly, the conditions for optimal transfection of nucleic acids into cells using the herein-mentioned procedures must be optimized. Such optimization is within the scope of one of ordinary skill in the art without the need for undue experimentation.

Dosages, Formulations and Routes of Administration of the Agents of the Invention

The agents of the invention are preferably administered so as to result in a reduction in at least one symptom associated with a disease. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems, which are well known to the art. As used herein, the term “therapeutic miRNA” refers to any miRNA that has a beneficial effect on the recipient. Thus, “therapeutic miRNA” embraces both therapeutic and prophylactic miRNA.

Administration of miRNA may be accomplished through the administration of the nucleic acid molecule encoding the miRNA. Pharmaceutical formulations, dosages and routes of administration for nucleic acids are generally known.

The present invention envisions treating LG in a mammal by the administration of an agent, e.g., a nucleic acid composition, an expression vector, or a viral particle of the invention. Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the eye. Alternatively the therapeutic agent may be introduced systemically (e.g., intravenously). The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules, as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0. saline solutions and water.

The invention will now be illustrated by the following non-limiting Example.

### Example 1

The present inventors have discovered that mir-184 is significantly down-regulated in corneal inflammatory LG, and accordingly, its synthetic mimic inhibits corneal lymphatic growth in vivo. Moreover, mir-184 overexpression in human lymphatic endothelial cells (LECs) in vitro suppresses their functions of adhesion, migration, and tube formation. These results together reveal that mir-184 is a negative regulator of the lymphangiogenic process.

Methods

Animals, Lymphatic Endothelial Cells and Reagents

Six to eight weeks old normal adult male BALB/c mice were purchased from Taconic Farms (Germantown, N.Y.). All mice were treated according to ARVO Statement for the Use of Animals in Ophthalmic and Vision Research and the protocols approved by the Animal Care and Use Committee of the institute. Mice were anesthetized using a mixture of ketamine, xylazine, and acepromazine (50 mg, 10 mg and 1 mg/kg body weight, respectively) for each surgical procedure. Human neonatal primary microdermal LECs were purchased from Lonza (Walkersville, Md.) and maintained in EGM-2MV cell culture medium (Lonza) according to manufacturer's instructions. Matrigel, collagen type I, and calcein AM were purchased from BD Biosciences (San Jose, Calif.). Mir-184 mimic and control RNA were purchased from Dharmacon, Inc. (Lafayette, Colo.) and Ambion (Austin, Tex.).

Induction of Corneal Lymphangiogenesis and Pharmaceutical Intervention

The experiments were performed as previously described (Grimaldo S, Yuen D, Ecoiffier T, Chen L. Very late antigen-1 mediates corneal lymphangiogenesis. Invest Ophthalmol Vis Sci 2011; 52:4808-4812; Yuen D, Grimaldo S, Sessa R, et al. Role of angiopoietin-2 in corneal lymphangiogenesis. Investigative ophthalmology & visual science 2014; 55:3320-3327). Standard suture placement model was used to induce corneal inflammatory LG. Briefly, three 11-0 nylon sutures (AROSurgical, Newport Beach, Calif.) were placed into the stroma of central corneas without penetrating into the anterior chamber. Mice were randomized to receive subconjunctival injections of either mir-184 mimic (10 μg; Dharmacon, Inc.) or control on day 0 and day 3 after suture placement. Experiments were repeated twice with a total of six mice in each group.

Immunofluorescent Microscopic Assay and Lymphatic Quantification

The experiments were performed as previously reported (Grimaldo S, Yuen D, Ecoiffier T, Chen L. Very late antigen-1 mediates corneal lymphangiogenesis. Invest Ophthalmol Vis Sci 2011; 52:4808-4812; Yuen D, Grimaldo S, Sessa R, et al. Role of angiopoietin-2 in corneal lymphangiogenesis. Investigative ophthalmology & visual science 2014; 55:3320-3327). Briefly, whole-mount corneas were sampled at one week after suture placement and fixed in acetone for immunofluorescent staining Lymphatic vessels were recognized by purified rabbit-anti-mouse LYVE-1 antibody, which was visualized by Cy3-conjugated donkey-anti-rabbit secondary antibody. Samples were covered with Vector Shield mounting medium (Vectashield; Vector Laboratories, Burlingame, Calif.). Digital images were taken with an epifluorescence microscope (AxioImager M1; Carl Zeiss AG, Göttingen, Germany) and analyzed using the NIH ImageJ software (provided in the public domain by the National Institutes of Health, Bethesda, Md., USA). The percentage scores of LG coverage areas were obtained by normalizing to control groups defined as being 100%. The differences were analyzed using Mann-Whitney test with p<0.05 as significant.

Reverse Transcription and Real-Time PCR

Total RNA from LECs or central corneal epithelium was extracted using miRNeasy Mini Kit (Qiagen, Valencia, Calif.). Reverse transcription was performed using the miScript II RT Kit (Qiagen). Real-time PCR was performed using miScript SYBR Green PCR Kit with specific primers to mature mir-184 using the mir-184_1 miScript Primer Assay (Qiagen) and measured by the CFX96 real time detection system (Bio-Rad Herecules, Calif.). Relative expression of the mir-184 was calculated from the δ-Ct (threshold cycle) of the targeted gene normalized to the δ-Ct of the RNU6B reference gene (Chien K H, Chen S J, Liu J H, et al. Correlation between microRNA-34a levels and lens opacity severity in age-related cataracts. Eye (Lond) 2013; 27:883-888). Traditional PCR products were also run on a 2% agarose gel.

Mir-184 Ectopic Expression in Lymphatic Endothelial Cells

Transfections were carried out with Lipofectamine RNAiMax (Invitrogen, Carlsbad, Calif.) according to manufacturer's instructions. As reported previously, the transfection of either mir-184 mimic or control RNA was carried out overnight at 37° C. in a 5% CO₂ humidified air incubator (Grimaldo S, Yuen D, Ecoiffier T, Chen L. Very late antigen-1 mediates corneal lymphangiogenesis. Invest Ophthalmol Vis Sci 2011; 52:4808-4812).

Adhesion Assay

The experiment was performed as previously described (Grimaldo S, Yuen D, Ecoiffier T, Chen L. Very late antigen-1 mediates corneal lymphangiogenesis. Invest Ophthalmol Vis Sci 2011; 52:4808-4812). Seventy-two hours following the transfection, 100 ft of cells (3×10⁵ cells/ml) were added to collagen type I-coated 96 plate wells and incubated for 30 minutes at 37° C. The plates were then washed several times and incubated with calcein (1 μg/ml) in hanks buffered salt solution (HBSS) for 30 minutes at room temperature. Plates were washed with PBS and fluorescent intensity from bound cells was measured with a Spectramax M5^{e} microplate reader (Molecular Devices, Sunnyvale, Calif.). Assays were performed in triplicate and repeated at least three times.

Migration Assay

Seventy-two hours following the transfection, a 200 μl pipette tip was used to create linear wounds within LEC monolayers. Phase images of the scratches were taken at time 0 and 29 hours using a Zeiss Axio Observer A1 inverted microscope (Carl Zeiss Inc., Germany) For better visualization of the scratch area by end of the study at 29 hours, the cells were stained with crystal violet. The TScratch program (Tobias Geback and Martin Michael Peter Schulz, Swiss Federal Institute of Technology, ETH Zurich, Zurich, Switzerland) was used to determine the percent of the open area (Geback T, Schulz M M, Koumoutsakos P, Detmar M. TScratch: a novel and simple software tool for automated analysis of monolayer wound healing assays. Biotechniques 2009; 46:265-274).

Tube Formation Assay

As previously reported, seventy-two hours following transfection, LECs were seeded (2×10⁴ cells/well) onto 96-well plates containing solidified matrigel and monitored for 24 hours under a Zeiss Axio Observer A1 inverted microscope (Carl Zeiss Inc., Germany) (Grimaldo S, Yuen D, Ecoiffier T, Chen L. Very late antigen-1 mediates corneal lymphangiogenesis. Invest Ophthalmol Vis Sci 2011; 52:4808-4812; Yuen D, Grimaldo S, Sessa R, et al. Role of angiopoietin-2 in corneal lymphangiogenesis. Investigative ophthalmology & visual science 2014; 55:3320-3327). Phase images of tubes were taken and total tube lengths were analyzed by NIH ImageJ software. Assays were performed in triplicate and repeated at least three times.

Statistical Analysis

The mean difference was analyzed by student t-test using Prism software (GraphPad, La Jolla, Calif.) unless otherwise indicated. The differences between the treatment and control groups were considered statistically significant when p<0.05.

Results

Mir-184 is Down-Regulated in Corneal Lymphangiogenesis

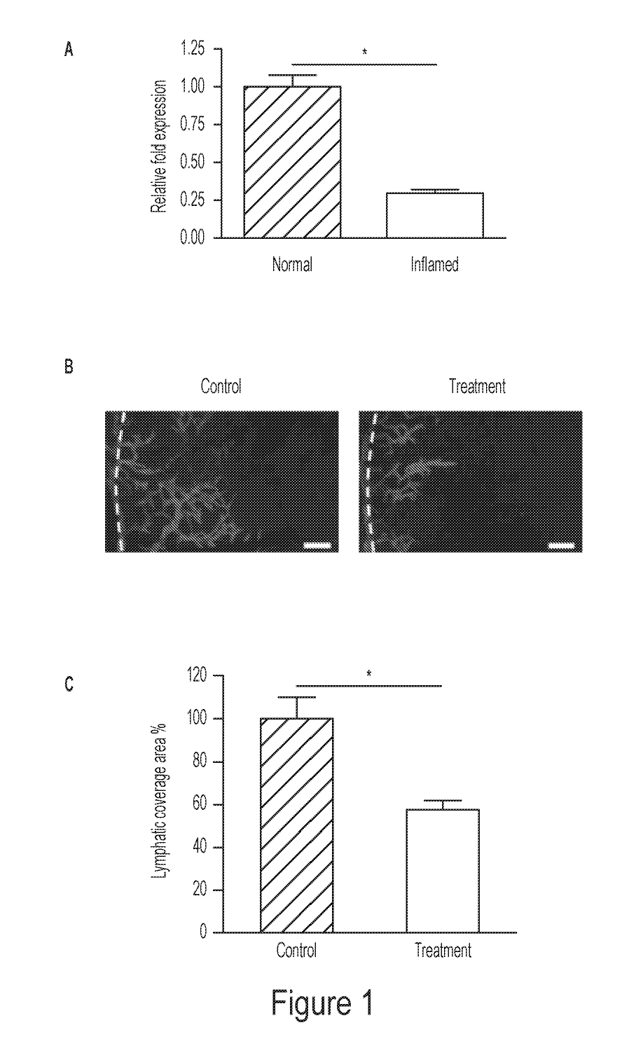

To investigate the role of mir-184 in corneal inflammatory LG, the expressional change of mir-184 in the inflamed cornea was first assessed following suture placement and lymphatic ingrowth. As shown in FIG. 1A by real-time PCR analysis, the expression level of mir-184 in the inflamed cornea was significantly down-regulated, as compared to the normal condition (* P<0.05). This result indicates that mir-184 may function as a natural inhibitor of corneal LG.

Synthetic Mimic of Mir-184 Suppresses Corneal Lymphangiogenesis In Vivo

To further explore whether mir-184 can be used as an inhibitor of corneal LG, the effect of mir-184 administration on inflammatory LG was assessed using synthesized mir-184 mimic, which acts to emulate the effect of mir-184. As shown in FIGS. 1B and 1C, the results from whole-mount corneal immunofluorescent microscopic analysis demonstrated that subconjunctival delivery of mir-184 mimic significantly reduced the lymphatic invasion area in the inflamed corneas (* P<0.05).

Ectopic Expression of Mir-184 in Lymphatic Endothelial Cells In Vitro

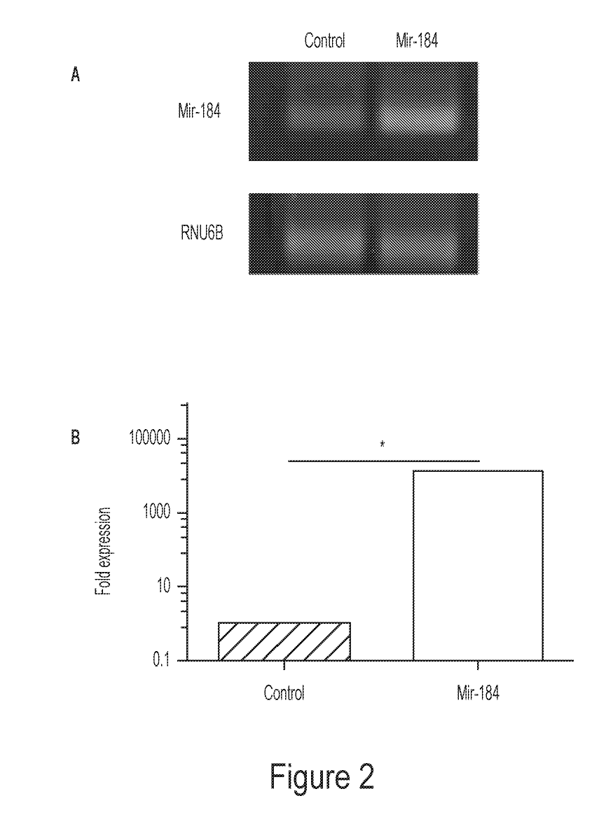

A human LEC culture system was used to study gain-of-function of mir-184 in vitro. To approach this, LECs were first transfected with mir-184 mimic, and enhanced expression of mir-184 was confirmed in these cells by both traditional and real-time PCR analysis (FIG. 2). As revealed by the agarose gel images in FIG. 2A, transfected LECs with mir-184 mimic showed an abundant PCR product corresponding to mature mir-184. FIG. 2B depicts the real-time PCR analysis confirming a significant fold increase of mir-184 expression in LECs after the transfection (* P<0.05). These results indicate that the approach is suitable to ectopically express mir-184 in LECs for further gain-of-function studies, as presented below.

Mir-184 Overexpression Inhibits Lymphatic Endothelial Cell Adhesion

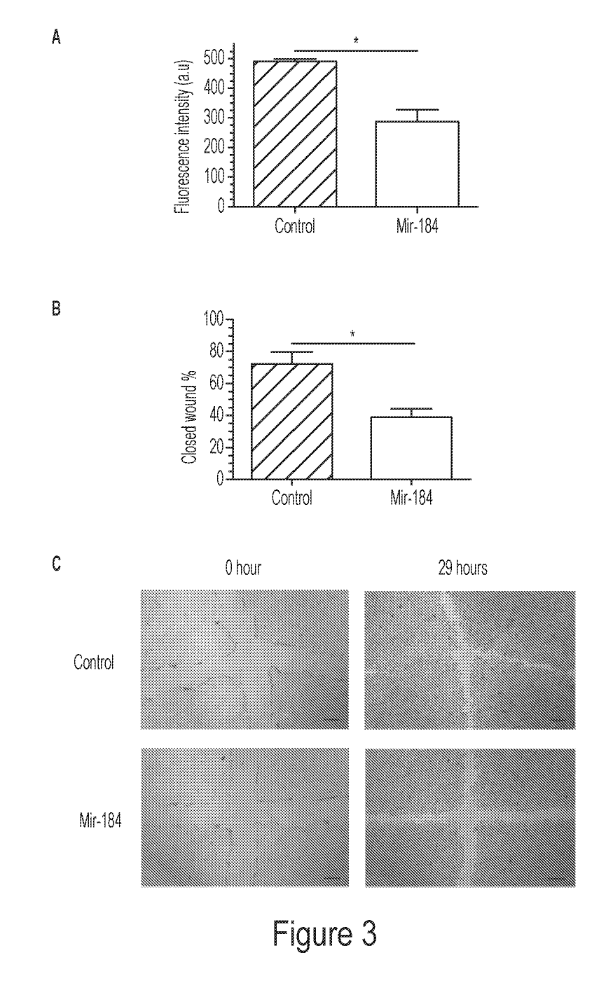

Adhesion is an important function of LECs in the lymphangiogenic process. whether mir-184 regulates LEC adhesion in vitro was determined. Seventy-two hours following the transfection with mir-184 mimic or control RNA, LECs were subjected to a collagen I adhesion assay, as reported previously (Grimaldo S, Yuen D, Ecoiffier T, Chen L. Very late antigen-1 mediates corneal lymphangiogenesis. Invest Ophthalmol Vis Sci 2011; 52:4808-4812). These results showed that mir-184 overexpression in LECs led to a significant reduction in cell adhesion (FIG. 3A, * P<0.05), suggesting a negative inhibitory role of mir-184 in this function.

Mir-184 Overexpression Reduces Lymphatic Endothelial Cell Migration

To assess whether mir-184 is also involved in LEC migration in intro, the wound healing scratch assay was performed seventy-two hours following the transfection with mir-184 mimic or control RNA. As presented in FIGS. 3B and 3C, mir-184 transfected cells showed a significant decrease in the rate of wound closing with larger open area (* P<0.05). These results indicate that mir-184 negatively regulates LEC migration as well.

Mir-184 Overexpression Suppresses Lymphatic Endothelial Cell Tube Formation

The effect of mir-184 overexpression on the ability of LECs to organize into capillary-type tubes using a three-dimensional (3D) culture system was examined. Seventy-two hours following the transfection with either mir-184 mimic or control RNA, LECs were seeded on matrigel, a basement membrane matrix, and observed for 24 hours. As shown in FIGS. 4A and 4B, mir-184 ectopic expression revealed a significant reduction in total tubule length (* P<0.05), confirming an inhibitory role of mir-184 in this important LEC function in vitro.

Discussion

In summary, this work provides the first evidence that mir-184 negatively regulates the LG process. Two important findings are reported: 1) mir-184 expression is significantly down-regulated in corneal inflammatory LG; and 2) mir-184 mimic can be used to suppress corneal LG in vivo and LEC functions in vitro. Taken together, this study not only divulges mir-184 as a suppressor of LG, but also put forth the use of mir-184 mimics as a novel strategy for LG therapy.

Mir-184 has a restrictive expression profile in the cornea, brain, and testes (Ryan D G, Oliveira-Fernandes M, Lavker R M. MicroRNAs of the mammalian eye display distinct and overlapping tissue specificity. Mol Vis 2006; 12:1175-1184; Karali M, Peluso I, Mango V, Banfi S. Identification and characterization of microRNAs expressed in the mouse eye. Invest Ophthalmol Vis Sci 2007; 48:509-515; Nomura T, Kimura M, Horii T, et al. MeCP2-dependent repression of an imprinted miR-184 released by depolarization. Hum Mol Genet 2008; 17:1192-1199). Previously reported microarray analysis revealed that it is one of the most abundantly expressed microRNAs in the corneal epithelium (Ryan D G, Oliveira-Fernandes M, Lavker R M. MicroRNAs of the mammalian eye display distinct and overlapping tissue specificity. Mol Vis 2006; 12:1175-1184). In the current study, it is shown that mir-184 is significantly down-regulated during corneal inflammatory LG; and it is highly indicated this microRNA may act to maintain the alymphatic status of the cornea under normal condition. Allied to this speculation is the additional data showing that re-introduction of mir-184 into the cornea suppresses lymphatic formation. As an immune privileged tissue, the cornea has developed mechanisms that actively maintain its lymphatic-free status under normal condition, which are yet to be fully explored and understood. Recently, it was reported that a soluble VEGFR-2 is secreted by the corneal epithelium and acts to suppress LG (Albuquerque R J, Hayashi T, Cho W G, et al. Alternatively spliced vascular endothelial growth factor receptor-2 is an essential endogenous inhibitor of lymphatic vessel growth. Nature medicine 2009; 15:1023-1030). This current study not only divulges mir-184 as a new inhibitor of LG, but also indicates that multiple mechanisms and factors are involved in maintaining the alymphatic status of the cornea.

It is known that Mir-184 mutation results in corneal pathologies in keratoconus and EDICT syndrome (Hughes A E, Bradley D T, Campbell M, et al. Mutation altering the miR-184 seed region causes familial keratoconus with cataract. Am J Hum Genet 2011; 89:628-633; Iliff B W, Riazuddin S A, Gottsch J D. A single-base substitution in the seed region of miR-184 causes EDICT syndrome. Investigative ophthalmology & visual science 2012; 53:348-353). To date, there has been no study linking mir-184 to corneal pathological LG. Previously, it was reported that mir-184 is involved in ischemia-induced retinal angiogenesis, and the downstream targets of mir-184 mediated angiogenesis are Wnt-receptor Frizzled 7 (Fzd7) and vascular endothelial growth factor-A (VEGF-A)(Shen J, Yang X, Xie B, et al. MicroRNAs regulate ocular neovascularization. Mol Ther 2008; 16:1208-1216; Takahashi Y, Chen Q, Rajala R V, Ma J X. MicroRNA-184 modulates canonical Wnt signaling through the regulation of frizzled-7 expression in the retina with ischemia-induced neovascularization. FEBS Lett 2015; 589:1143-1149). In the present study, a significant change in corneal angiogenesis as LG with mir-184 mimic treatment was not detected.

Research on mechanisms of corneal LG has broad implications. The use of synthetic microRNA mimic as a treatment, also known as “microRNA replacement therapy,” has emerged as a promising approach for disease treatment and has gained significant attention in cancer therapy (Bader A G, Brown D, Winkler M. The promise of microRNA replacement therapy. Cancer Res 2010; 70:7027-7030). MicroRNA replacement therapy focuses to reintroduce the microRNA in order to restore loss of function. In this study, using both in vivo murine LG model and in vitro human primary LEC culture system, a similar therapeutic approach is forth for LG interference so that mir-184 is used for the treatment of lymphatic-related diseases of the eye.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of suppressing pathological lymphatic formation in a tissue or organ, inhibiting cancer metastasis, and/or inhibiting transplant rejection, in a mammal in need thereof comprising administering an effective amount of a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic to the mammal.

2-3. (canceled)

4. A method of inhibiting adhesion, migration and/or tube formation of lymphatic endothelial cells (LECs) comprising administering to a mammal in need thereof an effective amount of a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic, wherein the inhibition is by about 10% as compared to non-treated LECs.

5. The method of claim 4, wherein the inhibition is by at least 40%.

6. A method of preventing or treating corneal lymphangiogenesis (LG) and/or modifying a cornea before or after transplantation to improve graft survival in a mammal in need thereof comprising administering an effective amount of a therapeutic agent comprising Mir-184, pri-Mir-184, a Mir-184 mimic, or a vector comprising an expression cassette comprising a promoter and a nucleic acid encoding Mir-184, pri-Mir-184, or a Mir-184 mimic to the mammal.

7. The method of claim 6, wherein the corneal lymphangiogenesis is induced by inflammation, infection, dry eye, trauma, or chemical damage.

8. (canceled)

9. The method of claim 1, wherein the tissue is eye tissue.

10. The method of claim 9, wherein the eye tissue is corneal tissue.

11. The method of claim 1, wherein the tissue is endothelial tissue.

12. The method of claim 11, wherein the endothelial tissue is lymphatic endothelial tissue.

13. The method of claim 1, wherein the mammal is a human.

14. The method of claim 1, wherein the therapeutic agent is present within a pharmaceutical composition.

15. The method of claim 1, wherein the administration is by local or systemic administration.

16. The method of claim 15, wherein the administration is by subconjunctival, intraocular, periocular, retrobulbar, intramuscular, topical, intravenous, or subcutaneous administration.

17. The method of claim 1, wherein the agent is a Mir-184 mimic that is 22 nucleotides in length.

18. The method of claim 1, wherein the agent is a pri-Mir-184 from 100-200 bp in length.

19. The method of claim 1, wherein the agent is a Mir-184 mimic that has at least 90% complementarity to SEQ ID NO: 1.

20. The method of claim 1, wherein the agent is a vector and the promoter is a polII or polIII promoter.

21. The method of claim 1, wherein the agent is a vector and the promoter is an H1 or U6 promoter.

22. The method of claim 1, wherein the agent is a vector and the promoter is a tissue-specific promoter.

23. The method of claim 1, wherein the agent is a vector and the promoter is an inducible promoter.

24. The method of claim 1, wherein the agent is an adeno-associated virus (AAV) vector or adenovirus vector.

25-27. (canceled)


SEQUENCE LISTING (2 sequences)

SEQ ID NO: 1 - RNA, 22 nt, Homo sapiens
uggacggagaacugauaagggu

SEQ ID NO: 2 - RNA, 84 nt, Homo sapiens
ccagucacguccccuuaucacuuuuccagcccagcuuugugacuguaaguguuggacggagaacugauaaggguaggugauuga